(19) United States
(12) Patent Application Publication
(10) Pub. No.: US 2024/0099643 A1
(43) Pub. Date: Mar. 28, 2024

(54) Systems and Methods of Cardiac Mapping

(71) Applicant: CATHETER PRECISION, INC., Fort Mill, SC (US)

(72) Inventor: David JENKINS, Flanders, NJ (US)

(21) Appl. No.: 18/370,670

(22) Filed: Sep. 20, 2023

Related U.S. Application Data
(60) Provisional application No. 63/409,964, filed on Sep. 26, 2022.
(60) Provisional application No. 63/431,821, filed on Dec. 12, 2022.

(51) Int. Cl. A61B 5/367; A61B 5/06; A61B 5/287

(52) U.S. Cl. CPC A61B 5/367; A61B 5/062; A61B 5/065; A61B 5/287

(57) ABSTRACT

A cardiac mapping method includes determining locations of contacts between a catheter and heart surfaces of a patient's heart using a localization system, selecting a three-dimensional (3D) heart model representative of the patient's heart based on the determined locations; using the localization system to detect locations of electrocardiogram (ECG) electrodes disposed on the patient to generate ECG electrode location data; collecting ECG data from the patient using the ECG electrodes; and generating an activation map of the heart based on the 3D model, the ECG data, and the location data. Embodiments also include ECG electrodes and intracardiac catheters that include an element configured to support localization by the localization system.

### CROSS-REFERENCE TO RELATED APPLICATIONS

This application claims the benefit of priority to U.S. Provisional Patent Application Ser. No. 63/431,821 filed Dec. 12, 2022, entitled “Systems and Methods of Cardiac Mapping” and U.S. Provisional Patent Application Ser. No. 63/409,964 filed Sep. 26, 2022, entitled “Systems and Methods of Cardiac Mapping,” the entirety of each of which is hereby incorporated herein by reference for all purposes.

### BACKGROUND

Heart defects in the cardiac conduction system can result in asynchronous contraction (arrhythmia) of the heart and are sometimes referred to as conduction disorders. As a result, the heart does not pump effectively, which may ultimately lead to heart failure. Conduction disorders can have a variety of causes, including age, heart (muscle) damage, medications and genetics.

Premature ventricular contractions (PVCs) are abnormal or aberrant heart beats that start somewhere in the ventricles rather than in the upper chambers of the heart as with normal sinus beats. PVCs typically result in a lower cardiac output as the ventricles contract before they have had a chance to completely fill with blood. PVCs may also trigger ventricular tachycardia (VT or V-Tach).

VT is another heart arrhythmia disorder caused by abnormal electrical signals in the ventricles of the heart. In VT, the abnormal electrical signals cause the heart to beat faster than normal, usually more than 100 beats per minute, with the beats originating in the ventricles. VT can occur in patients with structurally normal hearts caused by triggered or focal electrical activity. VT can also occur in patients with heart conditions such as myocardial scar caused by myocardial infarction and present as a re-entrant VT. One common location for idiopathic VT, (not myocardial scar related) is in the right ventricular outflow tract (RVOT), which is the route the blood flows from the right ventricle to the lungs. In patients who have had a heart attack, scarring from the heart attack can create a milieu of intact heart muscle and a scar that predisposes patients to develop VT.

### SUMMARY

Various embodiments provide cardiac mapping methods for generating three-dimensional (3D) representations of activation pathways of a patient's heart. Various embodiments may include locating positions, orientations and dimensions of the patient's heart structures by obtaining locations of electrocardiogram (ECG) electrodes positioned on the chest and a catheter positioned within the heart to facilitate, and using that information to select a suitable three-dimensional (3D) heart model for mapping activation pathways based on ECG data. Various embodiments may enable generating a 3D activation model of a patient's heart without the need for a separate imaging operation.

Various embodiments include cardiac activation mapping methods that use a localization system to determine several locations on endothelium and epithelium surfaces of a patient's heart touched with a tip or electrode of a catheter. The plurality of locations endothelium and epithelium surfaces may be determined in three-dimensions (3D) by the localization system, providing data that a processing system may use to select a representative 3D heart model of the patient's heart without the need for internal medical imaging. The localization system may be used to determine locations of electrocardiogram (ECG) electrodes disposed on the patient without the need for external imaging of the patient. ECG data may be obtained from the patient using the ECG electrodes. The processing system may then generate a 3D activation map of the heart based on the selected 3D heart model, the ECG data, and the determined locations of the ECG electrodes on the patient.

Various embodiments include cardiac mapping methods that involve applying ECG electrodes to a patient; using a localization system to detect locations of the ECG electrodes on the patient, using the localization system to locate a tip or electrode of a catheter where it is touching points on the patient's heart, using the determined locations of the ECG electrodes and touched points on the patient's heart to select a suitable 3D heart model, collecting ECG data from the patient using the ECG electrodes, and generating a 3D activation map of the heart based on the selected 3D heart model, the ECG data, and the locations of the ECG electrodes. Some embodiments include displaying the 3D activation map of the heart and performing a cardiac treatment procedure while viewing the displayed 3D activation map of the heart, such as performing an ablation procedure, implanting a pacemaker lead, implanting a defibrillator lead, and the like.

Further embodiments include ECG electrodes that include an element configured to support localization by a localization system. In some embodiments, the element included in the ECG electrodes includes one or more of a ferromagnetic material, a permanent magnet, an electromagnet that can be energized via an ECG lead, an ultrasound reflector, or an ultrasound transducer. Further embodiments include an intracardiac catheter that includes one or more elements configured to support localization by a localization system. In some embodiments, the element or elements included in the intracardiac catheter include one or more of a ferromagnetic material, a permanent magnet, an electromagnet that can be energized via an ECG lead, an ultrasound reflector, or an ultrasound transducer.

### BRIEF DESCRIPTION OF THE DRAWINGS

The accompanying drawings, which are incorporated herein and constitute part of this specification, illustrate example embodiments of the invention, and together with the general description given above and the detailed description given below, serve to explain the features of the invention.

FIG. 1 is a schematic representation of a cardiac mapping system, according to various embodiments.

FIG. 2A shows a three-dimensional (3D) heart model that may be generated by the system of FIG. 1.

FIG. 2B shows a 3D cardiac activation map that may be generated by the system of FIG. 1.

FIG. 3 shows a heart model that has been modified to include the localization point identified in the activation map according to various embodiments.

FIG. 4 is a schematic diagram illustrating major components of a system according to various embodiments.

FIG. 5A is a schematic diagram illustrating determining locations of ECG electrodes and a catheter tip using methods according to various embodiments.

FIG. 5B is a schematic diagram illustrating determining a plurality locations on the endothelium of a patient's heart using a multi-electrode intracardiac catheter according to various embodiments.

FIG. 5C is a schematic diagram illustrating a plurality locations on a patient's heart using methods according to various embodiments.

FIG. 6A is a process flow diagram illustrating operations of a method of generating a patient-specific 3D heart activation model according to various embodiments.

FIG. 6B is a flow diagram of images illustrating the operations of the method illustrated in FIG. 6A.

### DETAILED DESCRIPTION

Various embodiments will be described in detail with reference to the accompanying drawings. Wherever possible, the same reference numbers will be used throughout the drawings to refer to the same or like parts. References made to particular examples and embodiments are for illustrative purposes, and are not intended to limit the scope of the claims.

Various embodiments provide methods for generating a patient-specific three-dimensional (3D) activation model of the patient's heart without the need for internal and external imaging, thereby streamlining the procedures, reducing costs and time. Various embodiments include obtaining location and dimensional information of a patient's heart using localization methods that can determine the location of ECG electrodes on the patient and the tips and/or electrodes of a catheter inserted within the patient's heart as it contacts epicardium and endocardium surfaces, using this information to select a suitable 3D model of the heart, and locating the ECG electrodes with respect to the heart to support generating a patient-specific 3D activation model of the patient's heart. By obtaining localization information of the patient's heart and ECG electrodes on the patient's thorax, procedures to generate a 3D activation model of the patient's heart may be performed without the need for internal imaging of the heart (e.g., computerized tomography (CT) or magnetic resonance imaging (MRI)) and exterior imaging of the ECG electrodes (e.g., photographs of the patient with ECG electrodes attached). Further, such procedures may be performed at the same time as or just prior to the ablation or similar therapeutic treatment that is supported by the patient's 3D heart activation model. In this manner, the procedures required to obtain a patient's 3D heart activation model in support of ablation treatments may be streamlined, thereby reducing cost and time.

Catheter ablation is the treatment of choice in patients with VT and/or symptomatic PVCs. The targets for ablation are locations in the heart where the PVCs or VT's are occurring. In the case of an idiopathic VT the ablation site would be an arrhythmogenic foci. For scar related tachycardias, the VT would originate from an isthmus and the re-entrant loop would move (enter and exit) through the scar tissue.

Historically, a treating physician determined proper ablation locations by first stimulating or pacing the heart with an intracardiac catheter in a proposed location, to determine whether the location is close to the isthmus of the VT. If a desired activation pattern is not achieved when the heart is stimulated at a given location, a new location is chosen and the location stimulated or paced and the procedure repeated. Given limitations of this technique, new methods have been developed that can generate a 3D activation model of the patient's heart showing electrical pathways through cardiac tissues can provide improved guidance or determining proper ablation locations for treating ventricular arrhythmia and other cardiac conduction maladies.

Recently developed methods for generating patient-specific 3D heart activation models use a combination of internal and external imaging to obtain structure and location information regarding the patient's heart and ECG electrodes positions on the patient before ECG signals are collected. In particular, internal imaging, such as CT and/or MRI imaging, is one on a patient in order to obtain the 3D shapes and sizes of the patient's heart. This information is then used to select a particular 3D heart model from a library of such models that most closely matches the shape and size of the patient's heart thus is suitable for use in generating the 3D heart activation model based on ECG data. ECG electrodes are attached to the thorax of the patient and the locations of each ECG electrode are obtained by capturing an image of the patient with the electrode attached.

In such methods, a computer can correlate the ECG electrode locations on the patient to the selected 3D heart model by correlating the coordinate system internal and exterior imaging. When ECG data is collected by the ECG electrodes over multiple heartbeat cycles, the ECG signals gathered by each electrode in combination with the known location of each electrode are processed by the computer to map onto the selected 3D heart model how cardiac activation proceeds through the heart. The resulting output from the computer is a 3D representation of the patient's heart displayed on a monitor that shows how activation or ventricular depolarization wavefront passes through the heart tissues. The timing of depolarization across the heart may be represented with different colors or intensities which may appear as a regular shifting colors across the heart. Early or abnormal depolarization events occurring heart may appear as irregularities in the shapes and colors visible in the displayed heart model. Using the computer, a clinician can rotate an image of the 3D heart activation model to view the depolarization wavefront from various angles, including internal views of the heart.

The advantage of these new methods is that clinicians can identify arrhythmogenic foci, re-entrant loops and other irregular depolarization paths that are likely appropriate lesion targets in the heart in advance of conducting a cardiac ablation procedure. One drawback in methods is the extensive preparation time required to conduct the internal and external imaging before the ECG information can be collected.

An electrocardiogram (ECG) is defined herein as any method that (preferably non-invasively) correlates measured electrical activity of the heart muscle to measured or derived (electrical activity) of the heart. In the case of a classical electrocardiogram, the differences in potential between electrodes on the body surface are correlated to the electrical activity of the heart. In order to obtain such a functional image, an estimation of the movement of the electrical activity has to be provided.

Various embodiments include cardiac mapping methods for generating three-dimensional (3D) representations of activation pathways of a patient's heart that remove the requirement for internal and external imaging. Various embodiments may include locating positions, orientations and dimensions of the patient's heart structures by using a localization system to obtain locations of catheter electrodes or sensors positioned on and within the heart. In some embodiments, the localization system may track the location of catheter electrodes in real time, recording the location of any electrode touching a surface of the heart. This capability may enable obtaining several touch point locations on the heart by a clinician moving the catheter around within the heart to touch the endocardium in various locations or in the pericardial cavity between the parietal pericardium and the epicardium to touch epicardium in various locations. By identifying several locations on endocardium and epicardium touched by a catheter tip and/or electrodes, the size, shape and orientation of the patient's heart can be estimated by a computer. This information can be used by the computer to select a suitable 3D heart model for mapping activation pathways based on ECG data. The same localization system can identify the positions of the ECG electrodes on the patient including the positions with respect to the heart, with this information used by the computer to map ECG signals to heart structures for generating a 3D activation model of the heart. Various embodiments may enable generating a patient-specific 3D activation model of a patient's heart without the need for separate imaging operations.

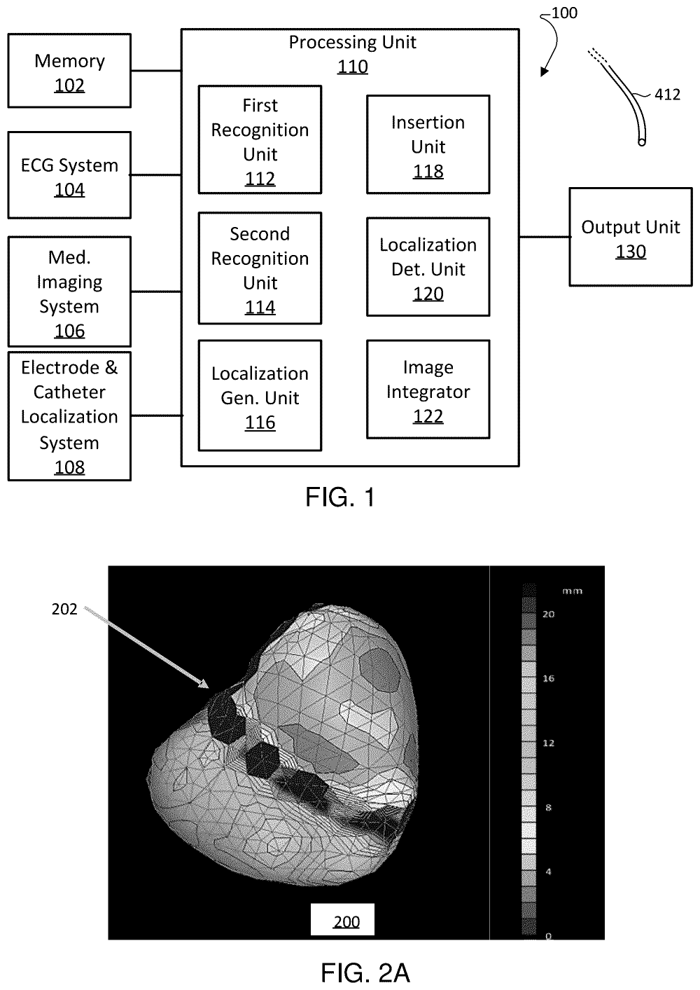

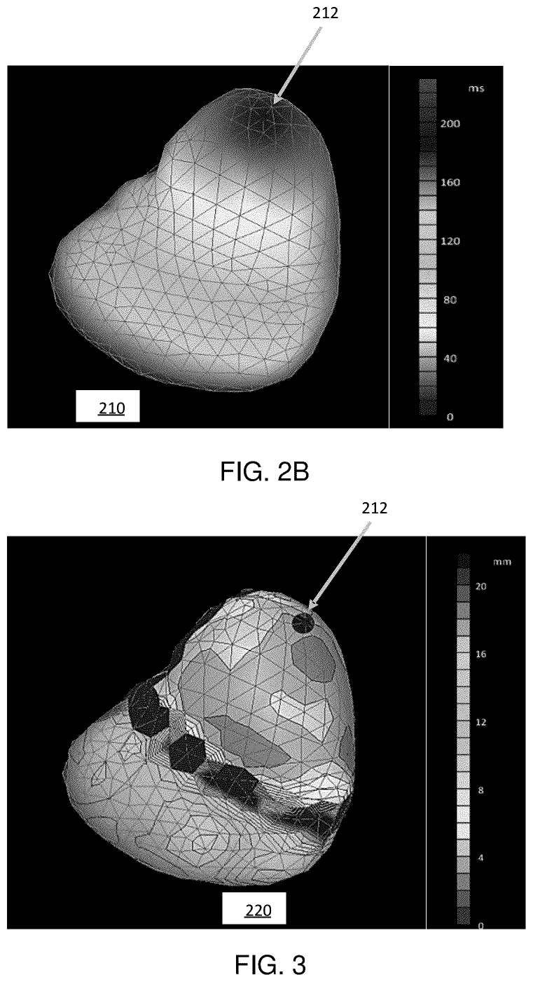

FIG. 1 is a schematic representation of a conventional cardiac mapping system 100 suitable for use with various embodiments. FIGS. 2A, and 2B illustrate conventional processes for generating a 3D activation model of the patient's heart. FIG. 2A shows a three-dimensional (3D) heart model 200 that may be generated by the system 100 of FIG. 1. FIG. 2B shows a 3D cardiac activation map 210 that may be generated by the system 100 of FIG. 1.

Referring to FIG. 1, the system 100 may include a processing unit 110, a memory 102, an optional output unit 130 (i.e., computer display), and a catheter 412 (e.g., an intracardiac catheter). The memory 102 may be configured to store computer-readable data and instructions. The output unit 130 may be a monitor or other display device. The processing unit 110 may include a central processing unit (CPU) or similar integrated circuit device, configured to execute instructions stored in the memory 102 or within the processing unit 110 to perform operations of various embodiments. The processing unit 110 may include a first recognition unit 112, a second recognition unit 114, a localization point generation unit 116, an insertion unit 118, a localization detection unit 120, and an image integrator 122.

The processing unit 110 may be configured to receive patient data from various sources, such as an electrocardiographic (ECG) system 104, a medical imaging system 106, and/or a localization system 108, and may be configured to store such data in the memory 102.

The processing unit 110 may be configured to select a 3D heart model 200 from a library or database of heart models using patient-specific measurements of the patient and the patient's heart generated by the medical imaging system 106. Conventionally, the medical imaging system 106 may be a magnetic resonance image (MRI) device, a computed tomography (CT) device, or the like. In various embodiments described herein, the medical imaging system 106 may be eliminated or unnecessary as measurements of the patient's heart may be made by determining the location of the tip or electrode of the catheter 412 when it is touched to various endothelium and epithelium surfaces of the heart, using the multiple location measurements to determine the size, shapes and orientations of various heart endothelium and epithelium surfaces. Based on location, orientation, size and configuration deals of the patient's heart obtained from internal imaging, a 3D anatomical heart model having the closest conformity to the patient's heart may be selected from a database of several 3D anatomical heart models. Such a database of 3D anatomical heart models may be representative of a variety of heart shapes and sizes, such as may be developed based on medical imaging of many patients and/or cadaver studies. In some embodiments, a selected 3D heart model may be modified to include some patient-specific features, such as particular structural dimensions, scar tissue, tissue thicknesses, etc. The selected and optionally modified 3D anatomical heart model may serve as the patient-specific 3D heart model 200. Such a model may include detailed structures of the heart such as the aortic cusps, aortic root, aorta, aortic arch, coronary vascular structures, or the like.

Conventionally, the 3D heart model 200 illustrated in FIG. 2A may be selected based on CT and/or MRI scans of the patient. This patient specific 3D heart model 200 may be generated by comparing the patient's digital imaging and communications in medicine (DICOM) standard images to reference 3D heart models obtained from the database of such models that may be stored in local memory or remote memory accessible via a network. The best fitting reference 3D heart model may be selected from the database and then adjusted (e.g., edited) to match to the patient's DICOM images. The edited patient-specific heart model may display the myocardial thickness of the patient's heart. For example, the 3D heart model 200 may include a color-coding scale to represent the myocardium thickness in millimeters. For example, red, orange and yellow colors may represent thinner myocardium and green to blue colors may represent thicker myocardium. The cardiac septum 202 may be represented by a darker-blue/black mid ventricular line.

Using images of the patient's torso, the processing unit 110 may determine the location of each ECG electrode placed on a patient's torso. By combining the ECG electrode locations determined based on images of the patient with internal imagery (e.g., CT and/or MRI) of the patient's heart, the processing unit 110 may determine the distance between each ECG electrode and the patient's heart. With this information, the processing unit 110 may be configured to correlate the signals from each ECG electrode spanning the QRS complex to locations on the patient's heart.

Having received ECG signals from each ECG electrode, the processing unit 110 may generate an activation map 210 by combining ECG signals with the location information of the ECG electrodes using the selected 3D heart model. The processing unit 110 may determine the localization of the ECG signals within the heart tissue based on applying and electrocardiographic imaging (ECGI) method to the 12 lead ECG data. The ECG signals may be combined with the selected patient-specific 3D anatomical model of the heart and torso to compute the positions of cardiac isochrones, which are lines of equal potential as a function of time during the QRS complex. As the cardiac activation proceeds, the wave front may be identified as a progression of colors for the duration of the QRS waveform. Such a color representation may provide a map of the activation time sequence during the QRS complex.

The 3D heart activation map 210 may include a localization point 212. As used herein, the term localization point may refer to an origin or exit site of an improper or a regular depolarization wave within the heart tissue. Localization points may identify an onset point of premature ventricular contraction (PVC) or an exit point of a VT beat. For example, the localization point 212 of FIG. 2B corresponds to the starting point of a single PVC. FIG. 2B also shows the duration of the associated QRS complex, which in the illustrated example continued for 230 milliseconds. The example localization point 212 is positioned on the epicardial surface of the heart myocardium. The scale provides the point of earliest activation (0 ms) and may be represented by a color, such as red.

FIG. 3 shows an example of a heart model 220 that has been modified to include the localization point 212 identified in the activation map 210. In particular, the localization point 212 may be superimposed on the patient specific 3D heart model 200 to form the modified heart model 220 including the myocardium's wall thickness. The heart model 220 may provide an accurate estimate of where and how much pressure should be applied to insert an ablation catheter within the ventricular myocardium so as to reach the localization point 212. This information may assist a physician in guiding an ablation catheter to the optimal therapy location. In the example illustrated in FIG. 3, the localization point 212 is identified on the endocardial surface of the right ventricle. Also, the right ventricle thickness is within the range of 3 mm to 6 mm, and the left ventricle is within the range of 8 mm to 14 mm. The localization point 212 is located within the basal lateral wall (i.e. closer to the valve plane) of the right ventricle.

While the foregoing methods are effective in identifying locations for therapeutic ablation treatments for arrhythmia and other cardiac activation maladies, the amount of imaging required to select an appropriate 3D heart model for the patient and align the selected model accurately with the positions of ECG electrodes on the patient at the time that ECG signals collected adds to the complexity, time and cost of these procedures. Various embodiments overcome this requirement by utilizing catheter localization systems to enable methods of characterizing sizes and locations of cardiac structures of the patient' heart during an intracardiac catheterization procedure, which may be part of the ablation therapy procedure. In this manner, prior internal imaging patient's heart may be avoided.

A variety of commercially available systems and methods are capable of determining the location of a device, such as an electrode on a catheter, within a patient using a variety of physical phenomenon. In various embodiments, any of these available localization techniques and systems may be used to localize the tip and/or any electrodes on an intracardiac catheter while a clinician positions the catheter on different endocardium and epicardium surfaces of the patient's heart. This localization information may then be used to determine dimensional information about the patient's heart that can then be used to select an appropriate 3D heart model from the database of heart models as described above. The same localization system and techniques may be used to also identify the locations of the ECG electrodes applied to the patient's torso with sufficient accuracy to obviate the need for conducting optical imaging of the patient for purposes of localizing the electrodes. Nonlimiting examples of physical phenomenon that can be used by localization systems in various embodiments include magnetic field strength sensors, electrical impedance sensors, electric field sensors, or ultrasound transducers as described in more detail below.

As a nonlimiting example, a localization system may include three or more magnetic field sensors that are configured to measure the magnetic field strengths resulting from interactions by ferromagnetic materials in electrodes on an intracardiac catheter as well as ECG electrodes. Example, the three or more localization system sensors may generate a magnetic field (e.g., a magnetic field of constant or varying strength) and measure the magnetic field at the sensors resulting from platinum electrodes on an intracardiac catheter. The resulting magnetic field strength measurable by the sensor will depend on the distance between each sensor and each electrode, strength of the generated magnetic field, the mass of the ferromagnetic material in the electrode, and the magnetic permeability characteristics of the intervening human tissues. As the strength of the generated magnetic field, the mass of the ferromagnetic material in the electrode, and the magnetic permeability characteristics of the intervening human tissues can be known, a computer can translate the magnetic field strength measurements into distances.

By using three or more such sensors, the location of each platinum electrode within a coordinate system defined by the sensors can be determined by the computer. The electrode positioned information can be determined in real time as each electrode moves within or outside the heart. Additionally, the positioning system can detect when any electrode contacts a surface of the heart (e.g., endocardium or epicardium) because there will be a drop in the resistance or reluctance through electrode contacting surface of the heart compared to the same electrode suspended in body fluids (e.g., blood). Using these capabilities, a positioning system according to various embodiments may automatically record a plurality of contact points on surfaces of the heart as a clinician moves the catheter about (within the heart or within the epicardial cavity layer) by tracking each electrode in real time and recording the location of any electrode in which the resistance or reluctance drops below a threshold value indicating contact with a heart surface and/or an ECG signal amplitude exceeds a threshold.

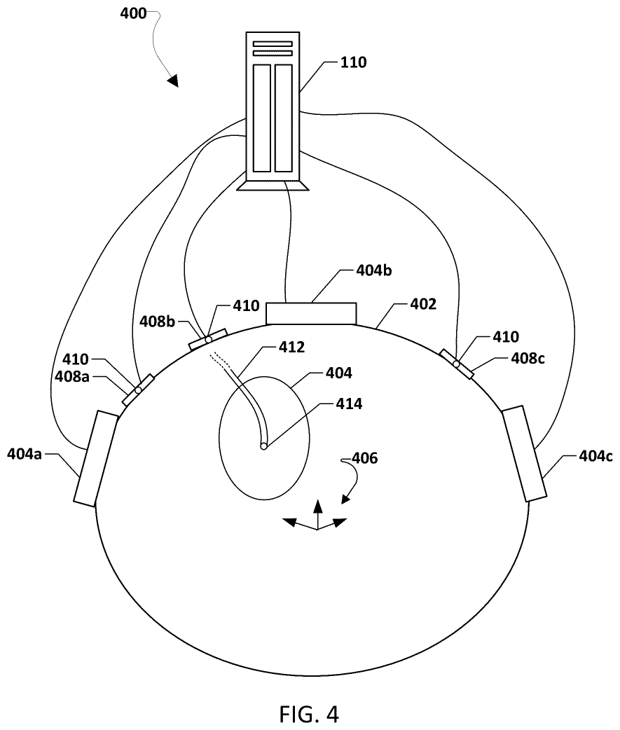

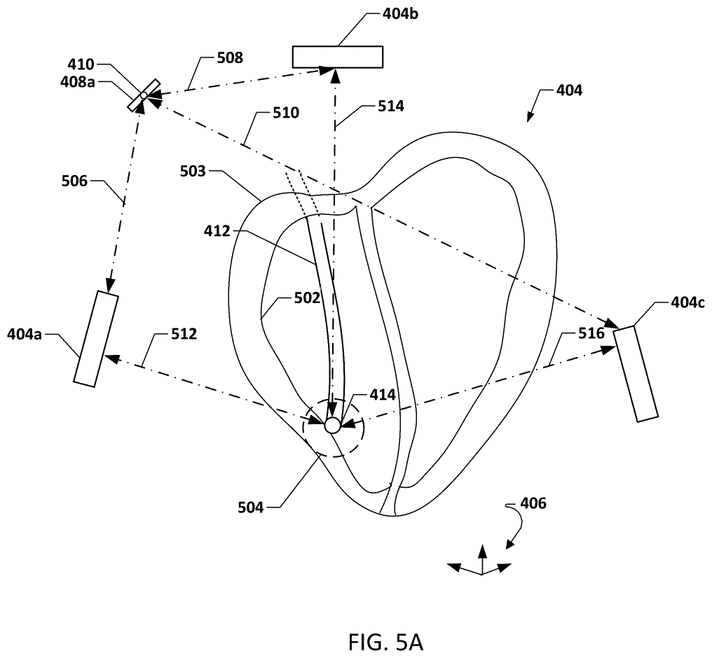

FIG. 4 is a schematic block diagram illustrating major components of a system 400 according to various embodiments. In this illustration, a patient's body 402 (e.g., chest) is shown in cross-section including a cross-section of the heart 404, with the electrode and catheter localization sensors 404a, 404b, 404c and ECG electrodes 408a, 408b, 408c applied to the outside of the chest. Also illustrated in FIG. 4 is an intra-cardiac catheter 412 positioned within the heart 404 for performing localization procedures according to various embodiments. The electrode and catheter localization sensors 404a, 404b, 404c and ECG electrodes 408a, 408b, 408c may be coupled via wired connections (as illustrated) or wireless communication links (e.g., Bluetooth) to a processing unit 110 such as illustrated in FIG. 1.

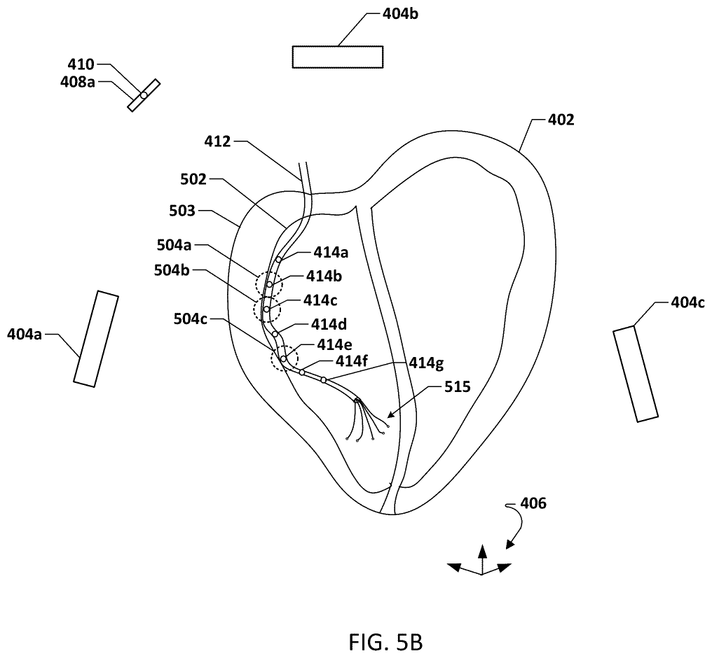

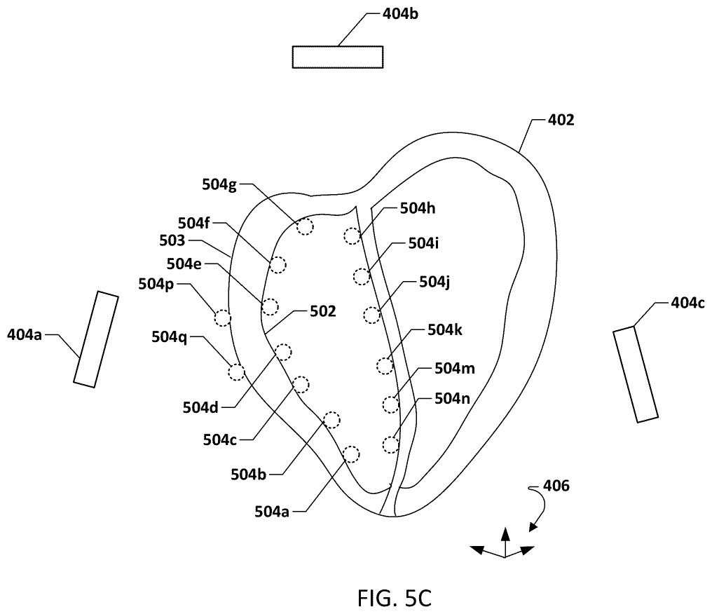

The electrode and catheter localization sensors 404a, 404b, 404c may be any form of sensors that can measure the distance to ECG electrodes (e.g., 408a, 408b, 408c) positioned on the patient's body 402 and the distance to a portion of an intra-cardiac catheter 412, such as a passive element (e.g., an electrode 414) or active element positioned on the tip. As explained in more detail with reference to FIGS. 5A and 5B, when the localization sensor 404a, 404b, 404c are placed in three spaced-apart locations on the patient's body 402, the distance measurements made by each sensor to each ECG electrode and to electrodes on the cardiac catheter 112 can be used by the processing unit 110 to identify 3D location coordinates of each ECG electrode and the tip and/or electrodes of the cardiac catheter 412 (e.g., an electrode 414 on the catheter tip) within a coordinate system 406 defined by the locations of the localization sensors 404a, 404b, 404c.

If the electrode and catheter localization sensors 404a, 404b, 404c remain in place during a catheterization procedure and are used to determine the location within the heart 104 of an ablation tip the catheter 412, then the coordinate system 406 defined by the locations of the localization sensors 404a, 404b, 404c can be used to correlate the location of the catheter ablation tip to the 3D activation heart model during the ablation procedure. In other words, it may not be necessary to determine the specific locations of each of the localization sensors 404a, 404b, 404c on the patient in order to correlate the catheter position to the 3D activation heart model and actual structures of the heart 404.

In some implementations, locations of the electrode and catheter localization sensors 404a, 404b, 404c on the patient 402 can be determined by photographic imaging for use in correlating the locations of electrodes and catheters to an external coordinate system. For example, the locations of the localization sensors 404a, 404b, 404c within an external coordinate system (e.g., with respect to an examination table, a catheter or surgical robot, or the catheterization laboratory) may be determined through image analysis by the processing system 110, with the determined sensor locations correlated with the patient's body or reference markers on the patient's body. In this manner, the processing system 110 may determine the catheter tip electrode 414 position (or any catheter electrode) within the heart 404 to be determined in the external coordinate system. Such procedures may be useful when ablation or other intracardiac procedures are performed using the generated 3D activation heart model sometime after the procedure in which the locations various heart structures are determined according to various embodiments, particularly when using robotic systems correlated to the external reference frame.

The electrode and catheter localization sensors 404a, 404b, 404c may be any form of active or passive sensor that is capable of measuring the distance separating each sensor and the ECG electrodes and/the electrodes of a catheter 412 (e.g., a tip electrode 414). In nonlimiting examples, the electrode and catheter localization sensors 404a, 404b, 404c may be magnetic field strength sensors, electrical impedance sensors, electric field sensors, or ultrasound transducers. In some embodiments, more than one type of sensor may be used to enable using two or more physical phenomenon for measuring the separation distances.

In embodiments in which the electrode and catheter localization sensors 404a, 404b, 404c are electric field sensors, the ECG electrodes 408a, 408b, 408c and the tip and/or electrodes of the catheter 412 may be configured to generate or interact with an electric field that is sensed by electrical impedance or electric field sensors within the localization sensors 404a, 404b, 404c.

In some embodiments in which the electrode and catheter localization sensors 404a, 404b, 404c measure electric fields, a voltage may be applied to each ECG electrode in sequence, with measurements made of the resulting electric field at each of the localization sensors 404a, 404b, 404c, with the measured electric field strengths provided to the processing system 106. The processing system 110 can then use the received electric field strength to estimate the separation distance between each sensor and each electrode based upon the measured electric field and the applied voltage using the inverse square law and known resistivity characteristics of the human body. A similar method may be a used to determine the location of the tip and/or electrodes of the catheter 412 by applying a voltage to the tip electrode 414 when it is touched to various heart structures. By determining the separation distance between each of the electrode and catheter localization sensors 404a, 404b, 404c and each of the ECG electrodes and catheter electrodes in this manner, the location of each can be determined within the ordinate system 406 defined by the sensor locations on the patient's body 402.

In some embodiments in which the electrode and catheter localization sensors 404a, 404b, 404c measure electric fields, a voltage pulse or oscillating electric field (e.g., a weak alternating current (AC) signal) may be applied to each ECG electrode in sequence with measurements made of the resulting electric field at each of the sensors, with the measured electric field strengths provided to the processing system 106. In some embodiments, the oscillating electric field may be at a radio frequency (RF) that will pass through human tissue. The processing system 110 can then use the received electric field strength to determine the reluctance (or RF attenuation characteristics) in the conduction path between each sensor and each electrode, and use the determined reluctance (or RF signal strength) to estimate the separation distance based on known characteristics of the human body. A similar method may be a used to determine the location of the tip and/or electrodes of the catheter 412 by applying a voltage pulse or AC current to the tip electrode and/or other electrodes of the catheter when touched to various heart structures. By determining the separation distance between each of the electrode and catheter localization sensors 404a, 404b, 404c and each of the ECG electrodes and catheter tip and/or electrodes in this manner, the location of each can be determined within the ordinate system 406 defined by the sensor locations on the patient's body 402.

In some embodiments, an element 410, 414 (referred to herein as a “signaling element”) that is configured to interact with the electromagnetic field or sound sensed or generated by the electrode and catheter localization sensors 404a, 404b, 404c may be included in the ECG electrodes 408a, 408b, 408c and the catheter 412 to facilitate distance measurements made by the system 400. The type of signaling element 410, 414 will depend upon the type of sensor used in the electrode and catheter localization sensors 404a, 404b, 404c.

In embodiments in which the electrode and catheter localization sensors 404a, 404b, 404c are magnetic field sensors, the signaling elements 410 may be a ferromagnetic material, such as a platinum electrode, that establishes a magnetic field around each electrode based on magnetic fields generated by the sensors 404a, 404b, 404c. Similarly, the signaling element in the catheter 412 may be a ferromagnetic material, such as a platinum electrode 414, that establishes a magnetic field around the catheter tip and/or electrodes based on magnetic fields generated by the sensors 404a, 404b, 404c. Each electrode and catheter localization sensors 404a, 404b, 404c may measure the magnetic field strength at its location and pass that information to the processing system 110. The processing system 110 can then use the received magnetic field strength to estimate the separation distance between sensor and magnet based upon the measured magnetic field, the strength of the magnetic fields generated by the sensors, and the mass of the ferromagnetic material using the inverse square law and known magnetic field absorption characteristics of the human body. To determine the location of each ECG electrode, measurements of the magnetic field strength at each of the electrode and catheter localization sensors 404a, 404b, 404c may be made as each electrode is attached to the patient's body 402. The magnitude of change of the magnetic field strength measured by each localization sensor as electrode is attached to the patient's body will change based on the mass of ferromagnetic material and the separation distances. Thus, magnetic field strengths measured for previously attached ECG electrode signaling elements 410 can be ignored when localizing newly attached ECG electrode. Similarly, the location of the ferromagnetic element on the tip and/or other electrode of the catheter 412 may be determined based on changes in the measured magnetic field strength measured by each of the electrode and catheter localization sensors 404a, 404b, 404c as the catheter is moved within or around the patient's heart 404. By determining the separation distance between each of the electrode and catheter localization sensors 404a, 404b, 404c and each of the ECG electrodes and catheter tip and/or electrodes in this manner, the location of each can be determined within the coordinate system 406 defined by the sensor locations on the patient's body 402.

In embodiments in which the electrode and catheter localization sensors 404a, 404b, 404c are magnetic field sensors, the signaling elements 410 may be small magnets that establish a magnetic field around each ECG electrode. Similarly, the signaling element 414 in the catheter 412 may be a small magnet that establishes a magnetic field around the catheter tip. Each electrode and catheter localization sensors 404a, 404b, 404c may measure the magnetic field strength at its location and pass that information to the processing system 110. The processing system 110 can then use the received magnetic field strength to estimate the separation distance between sensor and magnet based upon the measured magnetic field and the known magnetic strength of each signaling element 410, 412 using the inverse square law, the strength of magnetic fields and known magnetic field absorption characteristics of the human body. To determine the location of each ECG electrode, measurements of the magnetic field strength at each of the electrode and catheter localization sensors 404a, 404b, 404c may be made as each electrode is attached to the patient's body 402. The magnitude of change the magnetic field strength measured by each localization sensor as electrode is attached will change based on the strength of the magnet signaling element 410 and the separation distances. Thus, magnetic field strengths of previously attached ECG electrode signaling elements 410 can be ignored when localizing newly attached ECG electrode. Similarly, the lead location of the signaling element on the tip 414 and/or electrodes of the catheter 412 may be determined based on changes in the measured magnetic field strength measured by each of the electrode and catheter localization sensors 404a, 404b, 404c as the catheter is moved within the patient's heart 404. By determining the separation distance between each of the electrode and catheter localization sensors 404a, 404b, 404c and each of the ECG electrodes and catheter tip and/or electrodes in this manner, the location of each can be determined within the coordinate system 406 defined by the sensor locations on the patient's body 402.

In some embodiments, the signaling elements 410 in the ECG electrodes 408a, 408b, 408c may be electromagnets that can be turned on and off, such as via a voltage applied through the electrical connections to an ECG machine or the processing unit 110. In such embodiments, the location of each ECG electrode may be made after all electrodes have been attached to the patient's body 402. To do so, the electromagnet signaling elements 410 in of the ECG electrode 408a, 408b, 408c may be individually energized to generate a magnetic field, and the electrode and catheter localization sensors 404a, 404b, 404c may measure the resulting magnetic field strength and provide that information to the processing element 110, which can then estimate the separation distance between each localization sensor and each ECG electrode one at a time. That having determined the locations of all of the ECG electrodes 408a, 408b, 408c on the patient's body 402, the ECG electrode electromagnetic signaling elements 410 may remain deenergized so the only field strength measured by the electrode and catheter localization sensors 404a, 404b, 404c is that of a magnetic signaling element 414 on the tip and/or electrodes of the catheter 412. By determining the separation distance between each of the electrode and catheter localization sensors 404a, 404b, 404c and each of the ECG electrodes and catheter tip and/or electrodes in this manner, the location of each can be determined within the coordinate system 406 defined by the sensor locations on the patient's body 402.

In embodiments in which the electrode and catheter localization sensors 404a, 404b, 404c are sonic or ultrasonic transducers, the signaling elements 410, 414 may be structures that resonate at the frequency of sound committed by such transducers, and thus produce strong echoes. In such embodiments, each of the electrode and catheter localization sensors 404a, 404b, 404c may omit sound or ultrasound pulses that travel through the patient's body 402 and are reflected back to the transducer by the echoing signaling elements 410, 414. By timing the round-trip of ultrasound between pulse and echo, the distance between each transducer and each echoing signaling elements 410, 414 in the ECG electrodes and the catheter 412 can be estimated by the processing system 110 based on the known speed of sound (or ultrasound) through the human body. By determining the separation distance between each of the electrode and catheter localization sensors 404a, 404b, 404c and each of the ECG electrodes and catheter tip in this manner, the location of each can be determined within the coordinate system 406 defined by the sensor locations on the patient's body 402.

FIG. 5A illustrates further details of processes of determining the locations and dimensions of heart structures using various embodiments. As described above, the sensors in the electrode and catheter localization sensors 404a, 404b, 404c can measure fields or signals (e.g., magnetic field strength, electric fields, round trip time of reflected ultrasound, etc.) generated by or received from the ECG electrodes and the catheter that enables the processing system 110 to estimate the separation distances.

Referring to FIG. 5A for example, the coordinates of a ECG electrode 408a may be determined by measuring the distance 506 between a first electrode and catheter localization sensor 404a and the electrode, measuring the distance 508 between a second electrode and catheter localization sensor 404b and the electrode, and measuring the distance 510 between a third electrode and catheter localization sensor 404c and the electrode. As discussed above processing unit 110 may determine these measurements by processing measured parameters (e.g., magnetic field strength, electric field strength, round-trip time of ultrasound echoes, etc.) provided by the sensors in the electrode and catheter localization sensors 404a, 404b, 404c using known propagation characteristics of the human body.

Similarly, a contact location 504 of the catheter 412 tip electrode 414 on endothelium 502 or epithelium 503 cardiac structures may be determined by measuring the distance 512 between the first electrode and catheter localization sensor 404a and contact location 504, measuring the distance 514 between the second electrode and catheter localization sensor 404b and contact location 504, and measuring the distance 516 between the third electrode and catheter localization sensor 404c and contact location 504. As discussed above processing unit 110 may determine these measurements by processing measured parameters (e.g., magnetic field strength, electric field strength, round-trip time of ultrasound echoes, etc.) provided by the sensors in the electrode and catheter localization sensors 404a, 404b, 404c using known propagation characteristics of the human body. Measuring these distances 506-516, enables the processing system 110 to locate each ECG electrode 408a and catheter electrode 414 within a coordinate system 406 that is defined by the locations of the three or more electrode and catheter localization sensors 404a-404c.

FIG. 5B illustrates an intracardiac catheter 412 that includes multiple electrodes 414a-414g, 515 being used to with the positioning system using magnetic field sensors 404a-404c to identify the coordinate positions of multiple touch points 504a-504c on interior surfaces (i.e., endocardium 502) within a coordinate system 406 is defined by the three or more sensors 404a-404c.

Various embodiments may be used with a variety of intracardiac catheters, including catheters that have multiple electrodes (i.e., not just on the tip). For example, an intracardiac catheter may have multiple electrodes positioned along the length of the portion that will be position within the heart. As another example, some intracardiac catheters include multiple electrodes that can be extended from a tip of the catheter such as in a fan or tree of wires with an electrode on each wire tip. FIG. 5B illustrates examples of both types of multiple electrodes on an intracardiac catheter 412. Specifically, the intracardiac catheter 412 shown in the figure includes seven electrodes 414a-414g along a length of the catheter, as well as a fan of electrodes 515 extended from a tip of the catheter.

Using some embodiments, catheters configured with multiple electrodes may be used for localizing multiple touch points on the endothelium 502 or epithelium 503 of the patient's heart even though the clinician may not be able to directly control the positions of all electrodes. As described previously, localization system used for tracking the position of electrodes 414a-414g on the catheter 412 may track positions in real time, thus enabling the processing system 110 to have information on the location of all of the electrodes on the catheter at all times. Also as described previously, when an electrode touches a cardiac structure (i.e., endocardium 502 or epicardium 503), that contact may be recognized by the processing system 110 as a decrease in resistance or reluctance, such as a resistance/reluctance that is less than a predefined threshold and/or an ECG signal amplitude exceeds a threshold. Thus, as illustrated in FIG. 5B, the positioning system using magnetic field sensors 404a-404c that can sense the distance to platinum electrodes, may record the coordinates of touch points 504a, 504b, 504c of those catheter electrodes 414b, 414c, 414e that are in contact with a heart surface (e.g., the endocardium 502 as illustrated). This may enable the system to quickly collect position information on a large number of touch points on endothelium 502 or epithelium 503 surfaces as a clinician maneuvers the catheter 412 within the heart or the pericardial cavity. While FIG. 5B does not show any of the electrodes within the fan of electrodes 515 touching a cardiac surface, the same method may be applied for such electrodes.

Also, while FIG. 5B only shows the catheter 414 within the heart with electrodes 414b, 414c, 414e touching the endocardium 502, similar methods can be used when a clinician slips the catheter 414 into the pericardial cavity between the parietal pericardium and the epicardium 503 of the heart.

Also as illustrated in FIG. 5B, the ECG electrodes 408a-408c may include a ferromagnetic element 410, such as platinum that makes up the electrical contact. By simultaneously locating the ECG electrodes 408a and the multiple touch points 504a-504c within the coordinate system 406, the computer system can correlate the locations of each ECG electrode to the heart structures, thereby providing the information needed to generate the patient-specific 3D heart activation model.

FIG. 5C illustrates how these methods of locating contact locations 504 of a catheter tip and/or electrodes on endocardium 502 and epicardium 503 surfaces can be used to determine the locations, orientations, shapes and thicknesses of various heart structures to select an accurate 3D model of the patient's heart without MRI imaging. In particular, the methods described with reference to FIGS. 4, 5A and 5B may be performed to determine coordinates in the coordinate system 406 of contact locations or touch points 504a-504n by the clinician moving the catheter 412 to touch many locations on the heart the tip 414 and/or electrodes 414a-414g while the localization system tracks the electrodes and detects each touch, the processing system 110 may determine the coordinates of many touch points 504a-504n. Having defined the coordinates of heart surfaces, the processing system 110 has size, shape and orientation information for heart structures sufficient to select a suitable heart model for use in generating an activation heart model as described herein.

With the different separation distances 506-516 determined between each of the catheter electrodes and catheter localization sensors 404a, 404b, 404c and the various ECG electrodes 408a, 408b, 408c and each of the many touch points 504a-504q, the processing system 110 can determine the coordinate locations of each electrode and the catheter tip and/or electrodes within the coordinate system 406 defined by the locations on the body of the localization sensors. The coordinates in the coordinate system 406 of the ECG electrodes and each contact location 504a-504q of the catheter electrode(s) may be used by the processing system 110 for generating the activation heart model based on ECG measurements by the various ECG electrodes as described herein. If an ablation or other catheterization procedure is performed without removing the electrode and catheter localization sensors 404a, 404b, 404c, no further manipulation by the processing system 110 of the electrode and catheter coordinates may be required. Subsequent procedures may be planned that require localizing heart structures within an external coordinate system (e.g., with respect to an operating table, catheter or surgical robot, or operating room. To support such procedures, the processing system 110 can perform a simple coordinate transformation of electrode and catheter coordinates based on a coordinate relationship between the external coordinate system and the sensor-based coordinate system 406.

In addition to methods of generating a patient-specific heart activation map, various embodiments include ECG electrodes and intracardiac catheters that include elements that interact with the catheter electrodes and catheter localization sensors 404a, 404b, 404c to facilitate the measurements by the sensors used to locate the ECG electrodes and portions of catheters (e.g., electrodes). With reference to FIGS. 4, 5A and 5B, ECG electrodes and portions of intracardiac catheters may include an element 410, 414 that interacts with fields or signals generated by the localization sensors 404a, 404b, 404c, or generate fields or signals that can be sensed and measured by those sensors.

Some embodiments include ECG electrodes that include a ferromagnetic material element 410 that will interact with magnetic fields generated by catheter electrodes and catheter localization sensors 404a, 404b, 404c in a manner that the sensors can measure. In some embodiments, the ferromagnetic material element 410 may be a metal piece coupled or positioned adjacent to the conductive surface of the electrode. In some embodiments, the conductive surface of the electrode may be made of a ferromagnetic material element 410 of a size and/or configuration to be detectable by the catheter electrodes and catheter localization sensors 404a, 404b, 404c.

Some embodiments include ECG electrodes that include a permanent magnet element 410 of a size sufficient to generate a magnetic field that can be detected and/or measured by catheter electrodes and catheter localization sensors 404a, 404b, 404c. In some embodiments, the permanent magnet element 410 may be coupled or positioned adjacent to the conductive surface of the electrode. In some embodiments, the conductive surface of the electrode may be made of a magnetized metal of a size and/or configuration to be detectable by the catheter electrodes and catheter localization sensors 404a, 404b, 404c.

Some embodiments include ECG electrodes that include an electromagnet element 410 coupled or positioned adjacent to the conductive surface of the electrode that can be energized via an ECG lead to generate a magnetic field that can be detected and/or measured by catheter electrodes and catheter localization sensors 404a, 404b, 404c. In some embodiments, the electromagnet element 410 may be coupled or positioned adjacent to the conductive surface of the electrode. In some embodiments, the conductive surface of the electrode may be configured to function as an electromagnet when current is applied to the electrode. For example, an electrical lead may be coiled around a surface of the electrode. Such embodiment ECG electrodes may enable the catheter electrodes and catheter localization system to locate each electrode individually by energizing one ECG electrode electromagnet at a time, measuring the generated magnetic field in each sensor 404a, 404b, 404c to determine separation distances before energizing the next ECG electrode electromagnet.

Some embodiments include ECG electrodes that include an ultrasound reflector element 410 coupled or positioned adjacent to the conductive surface of the electrode. An ultrasound reflector may be a structure of a material and shape that exhibits a harmonic oscillation at a frequency matching or close to the frequency of ultrasound emitted by catheter electrodes and catheter localization sensors 404a, 404b, 404c that use ultrasound to locate ECG electrodes and intracardiac catheters.

Some embodiments include ECG electrodes that include an ultrasound transducer element 410 coupled or positioned adjacent to the conductive surface of the electrode that can be energized via an ECG lead to emit ultrasound that can be detected and/or measured by catheter electrodes and catheter localization sensors 404a, 404b, 404c. Such embodiment ECG electrodes may enable the catheter electrodes and catheter localization system to locate each electrode individually by energizing one ECG electrode ultrasound transducer at a time, measuring the ultrasound pulse time of arrival and/or amplitude in each sensor 404a, 404b, 404c to determine separation distances before energizing the next ECG electrode ultrasound transducer.

Some embodiments include an intracardiac catheter that includes a ferromagnetic material element 414 positioned on the catheter on or adjacent to the tip and/or electrodes. The ferromagnetic material element 410 may be configured to interact with magnetic fields generated by catheter electrodes and catheter localization sensors 404a, 404b, 404c in a manner that the sensors can measure. In some embodiments, the ferromagnetic material element 410 may be a metal piece coupled or positioned adjacent to the conductive surface of the electrode. In some embodiments, the conductive surface of the electrode may be made of a ferromagnetic material element 410 of a size and/or configuration to be detectable by the catheter electrodes and catheter localization sensors 404a, 404b, 404c.

Some embodiments include an intracardiac catheter that includes a permanent magnet element 414 positioned on the catheter on or adjacent to the tip and/or electrodes. The permanent magnet element 410 of a size sufficient to generate a magnetic field that can be detected and/or measured by catheter electrodes and catheter localization sensors 404a, 404b, 404c. In some embodiments, the permanent magnet element 414 may be coupled or positioned adjacent to the tip and/or electrodes. In some embodiments, the electrodes of the catheter may be made of a magnetized metal of a size and/or configuration to be detectable by the catheter electrodes and catheter localization sensors 404a, 404b, 404c.

Some embodiments include an intracardiac catheter that includes an electromagnet element 414 positioned on the catheter on or adjacent to the tip and/or electrodes that can be energized via an ECG lead to generate a magnetic field that can be detected and/or measured by catheter electrodes and catheter localization sensors 404a, 404b, 404c. In some embodiments, the electromagnet element 414 may be coupled or positioned adjacent to the conductive surface of the electrode. In some embodiments, the electrodes on such a catheter may be made configured to function as an electromagnet when current is applied to the electrode. For example, an electrical lead may be coiled around a surface of the electrode. Such embodiment catheter electrodes may enable the catheter electrodes and catheter localization system to locate each electrode individually by energizing one catheter electrode electromagnet at a time, measuring the generated magnetic field in each sensor 404a, 404b, 404c to determine the separation distances before energizing the next catheter electrode electromagnet.

Some embodiments include an intracardiac catheter that includes an ultrasound reflector element 414 positioned on the catheter on or adjacent to the tip and/or electrodes. An ultrasound reflector may be a structure of a material and shape that exhibits a harmonic oscillation at a frequency matching or close to the frequency of ultrasound emitted by catheter electrodes and catheter localization sensors 404a, 404b, 404c that use ultrasound to locate the tip and/or electrodes of an intracardiac catheter.

Some embodiments include an intracardiac catheter 412 that includes an ultrasound transducer element 414 positioned on the catheter on or adjacent to the tip and/or electrodes. The ultrasound transducer element(s) 414 can be energized via an electrical lead to emit ultrasound that can be detected and/or measured by catheter electrodes and catheter localization sensors 404a, 404b, 404c. Such embodiment intracardiac catheters 412 may enable the catheter electrodes and catheter localization system to locate each electrode on a catheter individually by energizing one electrode ultrasound transducer at a time, measuring the ultrasound pulse time of arrival and/or amplitude in each sensor 404a, 404b, 404c to determine the separation distances before energizing the next electrode ultrasound transducer.

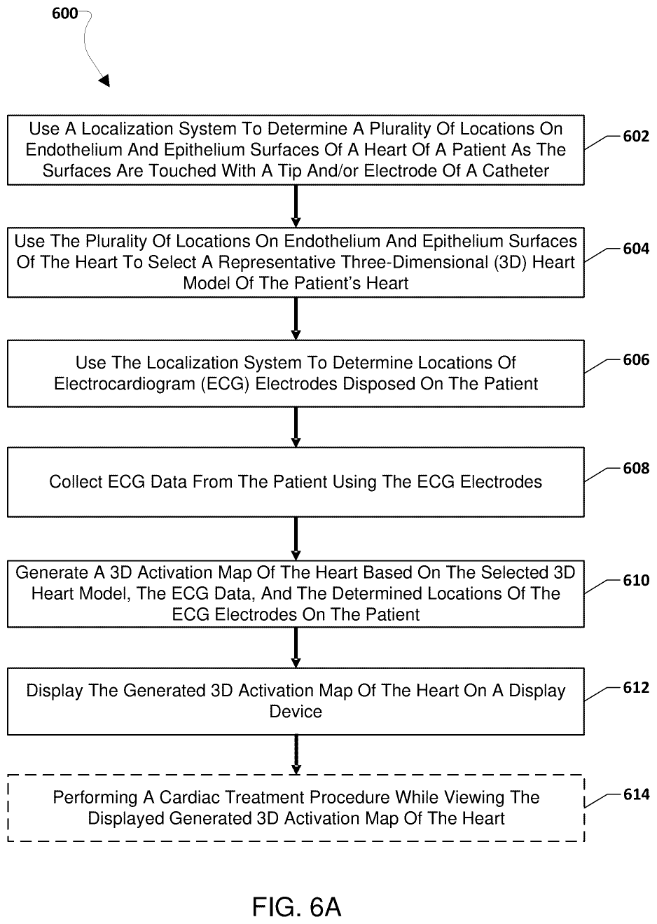

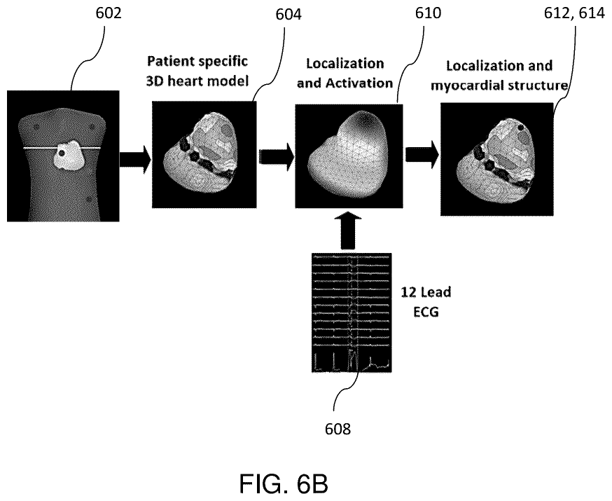

FIG. 6A is a process flow diagram illustrating operations of a method 600 of generating a patient-specific heart activation map according to various embodiments of the present disclosure. FIG. 6B is a flow diagram of images depicting the operations of the method 600 illustrated in FIG. 6A. The method 600 may be performed using the system 100 illustrated FIG. 1 with the exception that medical imaging system 106 may not be necessary. In particular, functions of the method 600 may be performed by a medical imaging system that includes a memory (e.g., 102), ECG electrodes (e.g., 408a-408c) that may include signal generators (e.g., 410) configured to generate location signals (either actively or passively), an intracardiac catheter (e.g., 412), a localization system (e.g., 108) that includes three or more sensors (e.g., 404a-404c) configured to detect the location signals and the location of a tip and/or electrodes (e.g., 414) of the intracardiac catheter when inserted in the heart; and a processing unit 110 coupled to the memory (e.g., 102), the ECG electrodes, and the localization system (e.g., 108) and configured with processor-executable instructions to perform operations of the method 600.

In block 602, the processing unit may perform operations including using a localization system to determine a plurality of locations on endothelium and epithelium surfaces of a heart of a patient as the surfaces are touched with a tip and/or electrode of a catheter. As described herein, the localization system may include three or more sensors that are configured to sense or measure physical fields or ultrasound that can be used by the processing unit to estimate the separation distances between the sensors and the tip and/or electrodes of the catheter. As explained in more detail herein, to estimate the separation distances, the processing unit may use measurements by the three or more sensors in combination with knowledge about the attenuation and/or transmission characteristics of human tissue for the physical field or ultrasound.

In an example implementation, in block 602 a clinician may manipulate an intracardiac catheter within the heart of the patient so as to touch the tip and/or electrodes against endothelium and epithelium surfaces of the heart while the processing system determines the location of the catheter tip and/or electrodes, such as in an X, Y, Z coordinate system, which may be defined by the three or more sensors of the localization system. By touching the endothelium and epithelium of the heart in multiple places while obtaining the three-dimensional locations of each touch point, the clinician in the system can build up a mesh of endothelium and epithelium surface locations defining the various structures of the heart. This manner, the imaging system can build up a 3D mesh of actual structures the patient's heart without the need for MRI or similar imaging.

In block 604, the processing unit may perform operations including using the plurality of locations on endothelium and epithelium surfaces of the heart to select a representative 3D heart model of the patient's heart. In some embodiments, the plurality of touch locations on the endothelium and epithelium of the heart may be used as a data base of locations or surface coordinates that can be matched up to various 3D heart models that may be maintained in a library or database of representative 3D heart models. For example, the processing unit may compare the plurality of touch locations to endothelium and epithelium dimensions of representative 3D heart model in the library or database and select the 3D heart model that most closely matches the majority of touch locations. In some embodiments, the processing unit may estimate the size, shape and orientation of heart structures from the plurality of locations on endothelium and epithelium surfaces of the heart determined based on distances from each of the three or more sensors to the signal generator on the tip and/or electrodes of the catheter when the tip and/or an electrode of the catheter touches an endothelium or epithelium surface of the heart. In such embodiments, a particular 3D heart model that best matches the estimated size, shape and orientation of heart structures may be selected from the library or database of 3D heart models. The various 3D heart models, and particularly the selected 3D heart model may include at least one heart structure selected from an aorta, an aortic arch, coronary vascular structures, pulmonary vascular structures, or scar tissue indicative of ischemic heart disease.

In block 606, the processing unit may perform operations including using the localization system to determine locations of ECG electrodes that have been disposed on (i.e., placed upon) the patient. The localization system may use the same physical mechanism for estimating the distance between the three or more sensors of the localization system and each of the ECG electrodes. In some embodiments, the ECG electrodes may include a material or device that either events or reflects a signal that can be sensed by the localization system sensors (referred to herein as a location signal), thereby enabling the same localization system that tracks the tip and/or electrodes of the catheter to also identify the locations of the ECG electrodes once they have been placed on the patient. The location of the ECG electrodes on the patient, and particularly with respect to the patient's heart, provides information that the processing unit can then use for correlating ECG signals to the progression of the activation wave through heart tissues as described herein.

The operations of measuring physical or ultrasound signals by the three or more sensor units of the localization system and estimating the distances between the sensors in each of the ECG electrodes and catheter tip and/or electrodes in block 602 and 606 will depend upon the physical phenomenon used for measuring the distances. For example, distance measurements may be based upon magnetic fields, electric fields (e.g., reluctance measurements) and ultrasound signals.

In some embodiments, the ECG electrodes and the tip and/or electrodes of the catheter may include structures that either emit measurable magnetic (e.g., permanent or electromagnets) or electric fields (e.g., a direct current (DC) voltage, and alternating current (AC) signal, or a radio frequency signal), with the sensors measuring the magnetic or electric field where the sensors are positioned on the patient. In some embodiments, the ECG electrodes and the tip and/or electrodes of the catheter may include an ultrasound emitting crystal that may be energized and configured to in emit pulses of ultrasound that can be received by the 3 or more sensors of the localization system, with either the sound intensity or the timing of received signals used for estimating the separation distance between the transducer and the receiving sensor. In some embodiments, the ECG electrodes and the tip and/or electrodes of the catheter may include a structure that resonates with and thus preferentially produces an ultrasound echo, with the three or more sensors of the localization system being ultrasound transducers that emit ultrasound and then measure the time delay for received echoes. In embodiments in which the ECG electrodes tip and/or electrodes the catheter include signal generators, the processing unit may use the localization system to determine the plurality of locations on endothelium and epithelium surfaces of the heart of the patient as the surfaces are touched with the tip and/or electrodes of the catheter in block 602 by determining distances from each of the three or more sensors to the signal generator on the tip of the catheter when the tip and/or electrodes of the catheter touches an endothelium and epithelium surface of the heart. Similarly, the processing unit may use the localization system to determine locations of the ECG electrodes disposed on the patient in block 606 by determining distances from each of the three or more sensors to the signal generator on each of the ECG electrodes.

In embodiments in which the three or more sensors of the localization system are magnetic sensors, the signal generators on the ECG electrodes and the tip and/or electrodes of the catheter may be magnets. In such embodiments, the processing unit may determine distances from each of the three or more sensors to the signal generator on each of the ECG electrodes and the tip o and/or electrodes of the catheter comprises measuring magnetic field strength by each of the three or more sensors and estimating the distances based on the measured magnetic field strength. The processing unit may use known magnetic field absorption and transmission characteristics of human tissue in estimating the separation distances based upon magnetic field strength measurements by the localization system sensors.

In embodiments in which the three or more sensors of the localization system are electric field sensors, the signal generators on the ECG electrodes and the tip and/or electrodes of the catheter may be electrodes. For example, the ECG electrodes may be configured to function as signal generators by being capable of applying a voltage, voltage pulse, AC signal or RF signal to the patient in an active mode in addition to receiving ECG signals in a passive (i.e., sensing) mode. In such embodiments, the processing unit may be configured to determine distances from each of the three or more sensors to the signal generator on each of the ECG electrodes and the tip and/or electrodes of the catheter by measuring electric fields by each of the three or more sensors and estimating the distances based on the measured electric fields. The processing unit may use known electric field and radio frequency absorption and transmission characteristics (e.g., reluctance, resistance, etc.) of human tissue in estimating the separation distances based upon electric field measurements by the localization system sensors.

In embodiments in which the three or more sensors of the localization system are ultrasound receivers or ultrasound transducers, the signal generators on the ECG electrodes and the tip and/or electrodes of the catheter may be ultrasound reflectors or ultrasound emitters. In such embodiments, the processing unit may be configured to determine distances from each of the three or more sensors to the signal generator on each of the ECG electrodes and the tip and/or electrodes of the catheter by measuring time delays between ultrasound pulses emitted by each of the three or more sensors and ultrasound echoes received from the ECG electrodes and the tip and/or electrodes of the catheter. The processing unit may use known sound transmission characteristics of human tissue (e.g., the speed of sound through various types of tissue) in estimating the separation distances based upon ultrasound pulse or echo timing measurements by the localization system sensors.

To enable determining the location of each of them multiple ECG electrodes, some embodiments may sequentially activate the signal generating structure in the ECG electrodes so that separation distances can be measured for each ECG electrode individually. In embodiments in which the three or more sensors of the localization system are magnetic sensors, the signal generators on the ECG electrodes may be electromagnets. In such embodiments, the processing unit may use the localization system to determine distances from each of the three or more sensors to the signal generator on each of the ECG electrodes in sequence by sequentially energizing the electromagnet in each ECG electrode while measuring magnetic field strengths by each of the three or more sensors of the localization system.

In block 608, the processing unit may perform operations including collecting ECG data from the patient using the ECG electrodes. Such operations may be consistent with conventional 12 lead ECG measurements, but may involve fewer or more leads depending upon the various implementations. In some embodiments, the processing unit may also receive ECG signals from electrodes 414a-414g, 515 on the intracardiac catheter 412 from within the heart, in which case the positioning system may also indicate the location within the heart of each catheter electrode providing ECG signals.

In block 610, the processing unit may perform operations including generating a 3D activation map of the heart based on the selected 3D heart model, the ECG data, and the determined locations of the ECG electrodes on the patient. As described above, such operations may involve mapping the ECG signals received from each electrode to locations on the 3D heart model based on timing and the distances between the heart structures and each ECG electrode (including electrodes on the intracardiac catheter if sampled). In some embodiments, the locations of the plurality of locations on the endothelium and epithelium surface of the heart and the determined locations of the ECG electrodes on the patient may be used by the processing unit to determine the distances between each ECG electrode and points on the 3D model of the heart, and generate the 3D activation map of the heart based on the distances between the electrodes in the heart. In some embodiments, the processing unit may modify the 3D heart model to include a PVC onset point detected using the ECG signal data, so that the PVC onset point can be displayed on the modified 3D heart model when presented on a monitor in block 612.

In the operations in block 610, the processing unit may apply a mathematical model (e.g., algorithm) to the ECG recording taken in block 608 using the ECG electrode locations and/or distances identified in in block 606. In some embodiments, the algorithm may be applied to ECG data corresponding to one or more PVC beats, or one or more VT beats, in order to generate an inverse solution that may be used to calculate one or more localization points. A cardiac activation map may be generated based on the patient specific heart model selected in block 604, with the map showing the propagation of electrical signals through the heart, including one or more localization points. For example, localization points may identify an earliest activation site, a latest activation site, a PVC onset point and/or a VT entry or exit point.

In block 612, the processing unit may generate a 3D activation map of the heart on a display device, such as on a monitor or computer display. The display may be integrated with user input devices, such as a computer mouse or trackball with the processing unit figured to rotate the 3D activation map of the heart in response to the user inputs so as to enable a clinician to view the activation sequence through heart tissues from various viewing angles. As described, this may enable a clinician to better prepare for and identify locations for conducting ablation treatments.

In optional block 614, a clinician may perform a cardiac treatment while viewing the display of the generated 3D activation map of the heart. In some embodiments, the cardiac treatment may be an ablation treatment, such as to treat arterial fibrillation. In some embodiments, the cardiac treatment may include placement of one or more pacemaker leads on the heart, and/or placement of one or more defibrillator leads on the heart. In some embodiments, the clinician may perform the cardiac treatment sometime after when the 3D activation map of the heart was generated, such as by displaying the 3D activation map of the heart downloaded from memory. In some embodiments, the clinician may perform the cardiac treatment at the same time or immediately following generation of the 3D activation map of the heart. For example, in the case of an ablation procedure, the clinician may use the same intracardiac catheter for identifying interior or anterior surfaces of the heart as for performing the ablation treatment.

It should be noted that the order of operations presented in FIG. 6 is for illustrative purposes and not intended to require a specific order of operations. In particular, the operations in block 602 through 608 may be performed in any order. For example, the operations in block 606 may be performed before or after the operations of locating a plurality of catheter tip and/or electrodes touch points on the endothelium and epithelium of the heart in block 602. Further, the collection of ECG data in block 608 may be performed before any of the operations in block 602 through 606 provided the operations in block 606 to localize the ECG electrodes are performed before the electrodes are removed from the patient.

Accordingly, the method of FIGS. 6A and 6B may allow for the generation of an activation map without the need for separate imaging procedures, such as an MRI, CT scan to identify cardiac structures, or an external torso scan to identify ECG electrode locations. As such, an activation map may be generated in real time, in a single procedure, which may be conducted in conjunction with or just prior to ablation therapies, thereby substantially reducing costs and procedure time.

The foregoing method descriptions and the process flow diagrams are provided merely as illustrative examples and are not intended to require or imply that the operations of the various embodiments must be performed in the order presented. As will be appreciated by one of skill in the art the order of operations in the foregoing embodiments may be performed in any order. Words such as “thereafter,” “then,” “next,” etc. are not intended to limit the order of the operations; these words are simply used to guide the reader through the description of the methods. Further, any reference to claim elements in the singular, for example, using the articles “a,” “an” or “the” is not to be construed as limiting the element to the singular.

The various illustrative logical blocks, modules, circuits, and algorithm operations described in connection with the embodiments disclosed herein may be implemented as electronic hardware, computer software, or combinations of both. To clearly illustrate this interchangeability of hardware and software, various illustrative components, blocks, modules, circuits, and operations have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

The hardware used to implement the various illustrative logics, logical blocks, modules, and circuits described in connection with the aspects disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but, in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. Alternatively, some operations or methods may be performed by circuitry that is specific to a given function.

In one or more exemplary aspects, the functions described may be implemented in hardware, software, firmware, or any combination thereof. If implemented in software, the functions may be stored as one or more instructions or code on a non-transitory computer-readable medium or non-transitory processor-readable medium. The operations of a method or algorithm disclosed herein may be embodied in a processor-executable software module and/or processor-executable instructions, which may reside on a non-transitory computer-readable or non-transitory processor-readable storage medium. Non-transitory server-readable, computer-readable or processor-readable storage media may be any storage media that may be accessed by a computer or a processor. By way of example but not limitation, such non-transitory server-readable, computer-readable or processor-readable media may include RAM, ROM, EEPROM, FLASH memory, CD-ROM or other optical disk storage, magnetic disk storage or other magnetic storage devices, or any other medium that may be used to store desired program code in the form of instructions or data structures and that may be accessed by a computer. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above are also included within the scope of non-transitory server-readable, computer-readable and processor-readable media. Additionally, the operations of a method or algorithm may reside as one or any combination or set of codes and/or instructions on a non-transitory server-readable, processor-readable medium and/or computer-readable medium, which may be incorporated into a computer program product.

The preceding description of the disclosed embodiments is provided to enable any person skilled in the art to make or use the present invention. Various modifications to these embodiments will be readily apparent to those skilled in the art, and the generic principles defined herein may be applied to other embodiments without departing from the scope of the claims. Thus, the present invention is not intended to be limited to the embodiments shown herein but is to be accorded the widest scope consistent with the following claims and the principles and novel features disclosed herein.

What is claimed is:

1. A cardiac activation mapping method, comprising:
using a localization system to determine a plurality of locations on endothelium and epithelium surfaces of a heart of a patient as the surfaces are touched with a tip or electrode of a catheter;
using the plurality of locations on endothelium and epithelium surfaces of the heart to select a representative three-dimensional (3D) heart model of the patient's heart;
using the localization system to determine locations of electrocardiogram (ECG) electrodes disposed on the patient;
collecting ECG data from the patient using the ECG electrodes; and
generating a 3D activation map of the heart based on the selected 3D heart model, the ECG data, and the determined locations of the ECG electrodes on the patient.

2. The method of claim 1, further comprising:
processing the plurality of locations on endothelium and epithelium surfaces of the heart and the determined locations of the ECG electrodes on the patient to determine distances between each ECG electrode and the heart; and
generating the 3D activation map of the heart further based on the distances between each ECG electrode and the heart.

3. The method of claim 1, wherein:
the ECG electrodes and the tip or electrode of the catheter comprise signal generators;
the localization system includes three or more sensors configured to receive signals from the signal generators;
using the localization system to determine the plurality of locations on endothelium and epithelium surfaces of the heart of the patient as the surfaces are touched with the tip or electrode of the catheter comprises determining distances from each of the three or more sensors to the signal generator on the tip or electrode of the catheter when the tip or an electrode of the catheter touches an endothelium and epithelium surface of the heart; and
using the localization system to determine locations of the ECG electrodes disposed on the patient comprises determining distances from each of the three or more sensors to the signal generator on each of the ECG electrodes.

4. The method of claim 3, wherein:
the three or more sensors of the localization system are magnetic sensors;
the signal generators on the ECG electrodes and the tip or electrode of the catheter are magnets or ferromagnetic material; and
determining distances from each of the three or more sensors to the signal generator on each of the ECG electrodes and the tip or electrode of the catheter comprises measuring magnetic field strength by each of the three or more sensors and estimating the distances based on the measured magnetic field strength.

5. The method of claim 3, wherein:
the three or more sensors of the localization system are electric field sensors;
the signal generators on the ECG electrodes and the tip or electrode of the catheter are electrodes; and
determining distances from each of the three or more sensors to the signal generator on each of the ECG electrodes and the tip or electrode of the catheter comprises measuring electric fields by each of the three or more sensors and estimating the distances based on the measured electric fields.

6. The method of claim 3, wherein:
the three or more sensors of the localization system are ultrasound transducers;
the signal generators on the ECG electrodes and the tip or electrode of the catheter are ultrasound reflectors; and
determining distances from each of the three or more sensors to the signal generator on each of the ECG electrodes and the tip or electrode of the catheter comprises measuring time delays between ultrasound pulses emitted by each of the three or more sensors and ultrasound echoes received from the ECG electrodes and the tip or electrode of the catheter.

7. The method of claim 3, wherein determining distances from each of the three or more sensors to the signal generator on each of the ECG electrodes comprises:
sequentially operating the signal generator in each ECG electrode to generate signals; and
using the localization system to determine distances from each of the three or more sensors to the signal generator on each of the ECG electrodes in sequence.

8. The method of claim 7, wherein:
the three or more sensors of the localization system are magnetic sensors;
the signal generators on the ECG electrodes are electromagnets; and
using the localization system to determine distances from each of the three or more sensors to the signal generator on each of the ECG electrodes in sequence comprises sequentially energizing the electromagnet in each ECG electrode while measuring magnetic field strengths by each of the three or more sensors of the localization system.

9. The method of claim 1, wherein using the plurality of locations on endothelium and epithelium surfaces of the heart to select the representative 3D heart model of the patient's heart comprises:
estimating size, shape and orientation of heart structures from the plurality of locations on endothelium and epithelium surfaces of the heart determined based on distances from each of the three or more sensors to the signal generator on the tip or electrode of the catheter when the tip or electrode of the catheter touches an endothelium and epithelium surface of the heart; and
selecting a 3D heart model from a database of 3D heart models that best matches the estimated size, shape and orientation of heart structures.

10. The method of claim 9, wherein the 3D heart model comprises at least one heart structure selected from an aorta, an aortic arch, coronary vascular structures, pulmonary vascular structures, or scar tissue indicative of ischemic heart disease.

11. The method of claim 1, further comprising:
generating the 3D activation map of the heart to include one or more of an earliest activation site, a latest activation site, a PVC onset point, a VT entry point or a VT exit point based on the ECG data; and
displaying the generated 3D activation map of the heart on a display device.

12. The method of claim 1, further comprising performing a cardiac treatment procedure while viewing the displayed generated 3D activation map of the heart.

13. The method of claim 12, wherein the cardiac treatment procedure comprises an ablation procedure.

14. The method of claim 12, wherein the cardiac treatment procedure comprises implanting a pacemaker lead or a defibrillator lead on the heart.

15. A medical imaging system comprising:
a memory;
electrocardiogram (ECG) electrodes comprising signal generators configured to generate location signals;
a localization system configured to detect the location signals and the location of a tip or electrode of an intracardiac catheter when inserted in the heart; and
a processing unit coupled to the memory, the ECG electrodes, and the localization system and configured with processor-executable instructions to perform operations comprising:
using the localization system to determine a plurality of locations on endothelium and epithelium surfaces of a heart of a patient as the surfaces are touched with the tip or electrode of the intracardiac catheter;
using the plurality of locations on endothelium and epithelium surfaces of the heart to select a representative three-dimensional (3D) heart model of the patient's heart;
using the localization system to determine locations of the ECG electrodes disposed on the patient;
collecting ECG data from the patient using the ECG electrodes; and
generating an activation map of the heart based on the selected 3D heart model, the ECG data, and the determined locations of the ECG electrodes on the patient.

16. The medical imaging system of claim 15, wherein:
the ECG electrodes and the tip or electrode of the intracardiac catheter comprise signal generators;
the localization system includes three or more sensors configured to receive signals from the signal generators; and
the processing unit is configured with processor-executable instructions to perform operations further comprising:
using the localization system to determine the plurality of locations on endothelium and epithelium surfaces of the heart of the patient as the surfaces are touched with the tip or electrode of the intracardiac catheter comprises determining distances from each of the three or more sensors to the signal generator on the tip or electrode of the intracardiac catheter when the tip or electrode touches an endothelium and epithelium surface of the heart; and
using the localization system to determine locations of the ECG electrodes disposed on the patient comprises determining distances from each of the three or more sensors to the signal generator on each of the ECG electrodes.

17. The medical imaging system of claim 16, wherein:
the three or more sensors of the localization system are magnetic sensors;
the signal generators on the ECG electrodes and the tip or electrode of the intracardiac catheter are magnets; and
the processing unit is configured with processor-executable instructions to estimate distances from the three or more sensors to the magnets in the ECG electrodes and the tip or electrode of the intracardiac catheter based on measurements of magnetic field strength by each of the three or more sensors.

18. The medical imaging system of claim 16, wherein:
the three or more sensors of the localization system are electric field sensors;
the signal generators on the ECG electrodes and the tip or electrode of the intracardiac catheter are electrodes; and
the processing unit is configured with processor-executable instructions to estimate distances from the three or more sensors to the ECG electrodes and the tip or electrode of the intracardiac catheter based on measurements of electric fields by each of the three or more sensors.

19. The medical imaging system of claim 16, wherein:
the three or more sensors of the localization system are ultrasound transducers;
the signal generators on the ECG electrodes and the tip or electrode of the intracardiac catheter are ultrasound reflectors; and
the processing unit is configured with processor-executable instructions to estimate distances from the three or more sensors to the ECG electrodes and the tip or electrode of the intracardiac catheter based on times between emitted ultrasound pulses and ultrasound echoes received from the ultrasound reflectors on the ECG electrodes and the tip or electrode of the intracardiac catheter.

20. The medical imaging system of claim 16, wherein the processing unit is configured with processor-executable instructions such that determining distances from each of the three or more sensors to the signal generator on each of the ECG electrodes comprises:
sequentially activating the signal generator in each ECG electrode to generate signals; and
using the localization system to determine distances from each of the three or more sensors to the signal generator on each of the ECG electrodes in sequence.

21. The medical imaging system of claim 20, wherein:
the three or more sensors of the localization system are magnetic sensors;
the signal generators on the ECG electrodes are electromagnets; and
the processing unit is configured with processor-executable instructions to sequentially energize the electromagnet in each ECG electrode while receiving magnetic field strength measurements from each of the three or more sensors of the localization system.

22. The medical imaging system of claim 20, wherein:
the three or more sensors of the localization system are electric field sensors;
the signal generators on the ECG electrodes are the electrodes coupled to a voltage source; and
the processing unit is configured with processor-executable instructions to sequentially energize each ECG electrode while receiving electric field measurements from each of the three or more sensors of the localization system.

23. The medical imaging system of claim 16, wherein the processing unit is configured with processor-executable instructions to perform operations such that using the localization system to determine the plurality of locations on endothelium and epithelium surfaces of a heart of the patient as the surfaces are touched with the tip or electrode of the intracardiac catheter comprises:
determining the location of the catheter tip or electrode in real time;
detecting contact of the catheter tip or electrode with a heart surface in a response to one or both of resistance or reluctance measured at the tip or electrode falling below a threshold or an ECG signal amplitude exceeds a threshold; and
recording the location of the catheter tip or electrode in response to detecting contact of the catheter tip or electrode with a heart surface.

24. A medical system, comprising:
means for using a localization system to determine a plurality of locations on endothelium and epithelium surfaces of a heart of a patient as the surfaces are touched with a tip or electrode of a catheter;
means for using the plurality of locations on endothelium and epithelium surfaces of the heart to select a representative three-dimensional (3D) heart model of the patient's heart;
means for using the localization system to determine locations of electrocardiogram (ECG) electrodes disposed on the patient;
means for collecting ECG data from the patient using the ECG electrodes; and
means for generating a 3D activation map of the heart based on the selected 3D heart model, the ECG data, and the determined locations of the ECG electrodes on the patient.
